(19) United States
(12) Patent Application Publication
(10) Pub. No.: US 2025/0360118 A1
(43) Pub. Date: Nov. 27, 2025

(54) TREATMENT OF FIBROTIC DISORDERS WITH METABOTROPIC GLUTAMATE RECEPTOR 5 ANTAGONISTS OR/AND CANNABINOID RECEPTOR 1 ANTAGONISTS

(71) Applicant: THE UNITED STATES OF AMERICA,AS REPRESENTED BY THE SECRETARY,DEPARTMENT OF HEALTH AND HUMAN SERVICES, Bethesda, MD (US)

(72) Inventors: Cinar Resat, Potomac, MD (US); Muhammad Arif, Rockville, MD (US); Abhishek Basu, Bethesda, MD (US); Malliga R. Iyer, Germantown, MD (US)

(21) Appl. No.: 18/873,511

(22) PCT Filed: Jun. 12, 2023
(86) PCT No.: PCT/US2023/025047

§ 371 Date: Dec. 10, 2024

Related U.S. Application Data
(60) Provisional application No. 63/352,428, filed on Jun. 15, 2022.

(51) Int. Cl. A61K 31/4439; A61K 31/404; A61K 31/415; A61K 31/4155; A61K 31/4168; A61K 31/437; A61K 31/44; A61K 31/506; A61K 31/519; A61P 11/00

(52) U.S. Cl. CPC A61K 31/4439; A61K 31/404; A61K 31/415; A61K 31/4155; A61K 31/4168; A61K 31/437; A61K 31/44; A61K 31/506; A61K 31/519; A61P 11/00

(57) ABSTRACT

The disclosure describes methods and compositions for reducing the level or activity of metabotropic glutamate receptor 5 (mGluR5) or/and cannabinoid receptor 1 (CB1R) for the treatment of fibrotic disorders such as pulmonary fibrotic disorders (e.g., fibrosing interstitial lung discases). In some embodiments, a mGluR5 antagonist or/and a CB1R antagonist (c.g., a peripheral CB1R antagonist) is/are used to treat a fibrotic disorder such as a pulmonary fibrotic disorder (e.g., a fibrosing interstitial lung disease).

### CROSS-REFERENCE TO RELATED APPLICATIONS

This application claims priority to/from and the benefit of U.S. Provisional Application No. 63/352,428 filed on Jun. 15, 2022, which is incorporated herein by reference in its entirety.

### STATEMENT OF GOVERNMENT SUPPORT

This invention was made with government support under grant number NIH Z01 AA000355-01 awarded by the National Institute on Alcohol Abuse and Alcoholism, National Institutes of Health.

### FIELD OF THE INVENTION

The present invention is directed generally to compositions and methods for the treatment of fibrotic disorders such as fibrosing pulmonary diseases (e.g., fibrosing interstitial lung diseases) by reducing the activity of cannabinoid receptor 1 (CB1R) or/and metabotropic glutamate receptor 5 (mGluR5).

### BACKGROUND OF THE INVENTION

Chronic pulmonary fibrosis results from scarring throughout the lungs which can be caused by many conditions including chronic inflammatory processes (e.g., sarcoidosis and Wegener's granulomatosis), infections, environmental agents (e.g., asbestos, silica and exposure to certain gases), exposure to ionizing radiation (such as radiation therapy to treat tumors of the chest), chronic conditions (e.g., lupus and rheumatoid arthritis), and even certain medications. In a condition known as hypersensitivity pneumonitis, fibrosis of the lungs can develop following a heightened immune reaction to inhaled organic dusts or occupational chemicals. This condition most often results from inhaling dust contaminated with bacterial, fungal, or animal products. In some types of pulmonary fibrosis, such as nonspecific interstitial pneumonitis (NSIP), the subject may respond to immunosuppressive therapy. Where, as in many cases, chronic pulmonary inflammation and fibrosis develop without an identifiable cause, the subject suffering from the disease often will not respond to medical therapy. This is particularly true of subjects suffering from idiopathic pulmonary fibrosis (IPF). The treatment options for idiopathic pulmonary fibrosis are very limited. There is no evidence that any medications can help this condition since scarring is permanent once it has developed. Lung transplantation is the only therapeutic option available in the majority of fibrosing interstitial lung diseases (ILDs).

Research trials using different drugs that may reduce fibrous scarring are ongoing. Since some types of lung fibrosis can respond to corticosteroids (such as prednisone) and/or other medications that suppress the body's immune system, these types of drugs are sometimes prescribed in an attempt to decrease the processes that lead to fibrosis. Nevertheless, it is well-recognized that at present there are no truly effective treatments for fibrosing diseases. It is a standard clinical practice to give patients prednisone and azathioprine, but there is no data showing that these drugs provide significant therapeutic benefit. In fact, the side-effects of these drugs may contribute to mortality in usual interstitial pneumonia (UIP) patients. Moreover, recently approved medications for idiopathic pulmonary fibrosis such as pirfenidone and nintedanib are not sufficient to fully attenuate the progression of fibrosis.

Therefore, there is an unmet need to identify therapeutic targets and effective therapeutic agents for the treatment of fibrotic disorders including pulmonary fibrotic disorders (e.g., fibrosing interstitial lung diseases).

### SUMMARY OF THE INVENTION

The present disclosure addresses the need for effective medications for treatment of fibrotic disorders including fibrosing lung diseases. The disclosure provides antagonists of metabotropic glutamate receptor 5 (mGluR5) or peripheral antagonists of cannabinoid 1 receptor (CB1R), or a combination thereof, for the treatment of fibrotic disorders including fibrosing pulmonary diseases.

In one aspect, the present invention is directed to methods for reducing fibrosis in a fibrotic disorder such as a pulmonary fibrotic disease, comprising decreasing the activity of cannabinoid receptor 1 (CB1R) or/and the activity of metabotropic glutamate receptor 5 (mGluR5) in fibrocytes or/and fibroblasts at a fibrotic lesion. Fibrotic lesions in an organ such as the lungs are areas of scarring of tissues of the organ such as the lung tissues.

In another aspect, the present invention is directed to methods for preventing, inhibiting the development of, treating, ameliorating, slowing, or reducing one or more symptoms of, or reversing the condition of, or otherwise achieving a therapeutic outcome, of a fibrotic disorder such as a pulmonary fibrotic disease in a subject, comprising administering to the subject a therapeutically effective amount of a composition comprising a metabotropic glutamate receptor 5 (mGluR5) antagonist or/and a therapeutically effective amount of a composition comprising a peripheral cannabinoid receptor 1 (CB1R) antagonist. In a combination therapy, a mGluR5 antagonist and a peripheral CB1R antagonist can be provided in the same composition or in separate compositions.

A better understanding of features and advantages of the present disclosure will be obtained by reference to the following detailed description, which sets forth illustrative embodiments of the disclosure, and the accompanying drawings.

### BRIEF DESCRIPTION OF THE DRAWINGS

The following figures are exemplary embodiments wherein the like elements are numbered alike.

FIGS. 1A and B show that the expression of mGluR5 and the levels of glutamate increased in bleomycin-induced fibrotic lungs in mice. (A) Levels of glutamate in lungs. (B) Gene expression of Grm5, which encodes mGluR5. Data represent mean±S.E.M. n=4 for control, n=5 for bleomycin group. Data were analyzed by T-test. * (P<0.05) indicates statistical significance compared to the control group.

FIG. 2A-C show that the deletion of mGluR5 prevented mortality and fibrosis in bleomycin-induced pulmonary fibrosis (PF) in mice. (A) Generation of mGluR5 knockout (KO) mice and study design. (B) Survival curve. (C) Level of hydroxyproline as a fibrosis marker. Data represent mean±S.E.M. n=4 for control, n=5 for 14 days post-bleomycin group, n=4 for 28 days post bleomycin group. Data were analyzed by two-way ANOVA followed by Sidak's multiple comparisons test. * (P<0.05), ** (P<0.01), *** (P<0.001), and * (P<0.0001) indicate statistical significance.

FIG. 3A-G show that the deletion of mGluR5 prevented bleomycin-induced decline in pulmonary function. (A) Pressure-Volume curve, (B) Tissue elasticity, (C) Peripheral airway resistance, (D) Forced vital capacity, (E) Forced expiratory volume (FEV), (F) Inspiratory capacity, (G) Inhaled air amount, as measures of lung function in wt (wild-type) and mGluR5 KO mice in control and 14 and 28 days after 1 U/kg single dose of oropharyngeal bleomycin. Data represent mean±S.E.M. n=4 for control, n=4 for 14 days post-bleomycin group, n=6 for 28 days post-bleomycin group. Data were analyzed by two-way ANOVA followed by Sidak's multiple comparisons test. * (P<0.05), ** (P<0.01), *** (P<0.001), and **** (P<0.0001) indicate statistical significance.

FIGS. 4A and B show that pharmacological inhibition of mGluR5 by CTEP prevented mortality. (A) Experimental design, (B) Survival curve. Data represent mean±S.E.M. n=8 for Bleomycin+Vehicle, n=8 for Bleomycin+CTEP (3 mg/kg). Mice were treated either vehicle or CTEP for 14 days by daily oral gavage.

FIG. 5A-C show that pharmacological inhibition of mGluR5 by fenobam attenuated pulmonary fibrosis and the decline in pulmonary function in a bleomycin-induced PF mouse model. (A) Experimental design, (B) Level of hydroxyproline in lungs, (C) Forced expiratory volume. Data represent mean±S.E.M. n=4 for control, n=6 Bleomycin+vehicle group, n=6 for Bleomycin+Fenobam (30 mg/kg) group. Mice were treated with either vehicle or fenobam for 14 days by daily oral gavage. Data were analyzed by one-way ANOVA followed by Dunnett's multiple comparisons test. * (P<0.05) indicates statistically significant difference.

FIG. 6A-C show that pharmacological inhibition of mGluR5 by basimglurant attenuated bleomycin-induced PF development in mice. (A) Survival curve, (B) % Body weight, (C) Pulmonary function parameter in vehicle- or basimglurant (3 mg/kg, PO)-treated wt mice 28 days after OP-Bleo. Treatments were performed between post-bleomycin day 8 and day 28. **** P<0.0001 indicates statistically significant difference.

FIG. 7 shows that the deletion of CB1R in mice attenuated bleomycin-induced alterations in transcriptomics clusters 0, 1, 2, 3, and 4 while the deletion of mGluR5 prevented alterations in clusters 5 and 6. Left part of FIG. 7: Total number of differentially expressed genes showed significantly lower alterations in CB1R knockout mouse compared to wild-type after the induction of PF using bleomycin. Middle part of FIG. 7: Transcriptome-wide co-expression networks analysis data of the bleomycin-induced PF mouse model identified 7 distinct clusters, with clusters 0, 1, and 4 significantly up-regulated and clusters 2 and 3 down-regulated after the genetic deletion of CB1R in bleomycin-induced PF mice. Right part of FIG. 7: 408 out of 470 genes from clusters 5 and 6, which were unchanged after CB1R deletion, were significantly up-regulated after the genetic deletion of mGluR5 in bleomycin-induced PF mice.

FIG. 8 shows that genes belonging to clusters 5 and 6 in murine lungs are similarly regulated by pulmonary fibrosis in humans. 214 out of 408 genes from clusters 5 and 6 affected by mGluR5 deletion in bleomycin-induced PF mice showed a similar transcriptional profile as late-stage human IPF patients.

### DETAILED DESCRIPTION OF THE INVENTION

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein, including substances and methods similar or equivalent to those described herein, may be employed in practicing the disclosure. It is also understood that every embodiment of the disclosure may optionally be combined with any one or more of the other embodiments described herein which are consistent with that embodiment.

It is further understood that the present disclosure encompasses analogs, derivatives, prodrugs, salts, solvates, hydrates, clathrates and polymorphs of all of the compounds/substances disclosed herein, as appropriate. The specific recitation of “analogs”, “derivatives”, “prodrugs”, “salts”, “solvates”, “hydrates”, “clathrates” or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned without recitation of any of these forms.

It is also understood that the present disclosure encompasses all possible stereoisomers, including all possible diastereomers and enantiomers and racemic mixtures of enantiomers, of the compounds/substances described herein, and not only the specific stereoisomers as indicated by drawn structure or nomenclature. Some embodiments of the disclosure relate to the specific stereoisomers indicated by drawn structure or nomenclature. The specific recitation of the phrase “or stereoisomers thereof” or the like with respect to a compound/substance or a group of compounds/substances in certain instances of the disclosure shall not be interpreted as an intended omission of any of the other possible stereoisomers of the compound/substance or the group of compounds/substances in other instances of the disclosure where the compound/substance or the group of compounds/substances is mentioned without recitation of the phrase “or stereoisomers thereof” or the like.

### Terminology

Unless defined otherwise or clearly indicated otherwise by their use herein, all technical and scientific terms used herein have the same meaning as commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

The compositions, methods, and articles can alternatively comprise, consist of, or consist essentially of, any appropriate materials, steps, or components disclosed herein. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints and all intermediate values of the ranges, and the endpoints are independently combinable with each other (e.g., ranges of “up to 25 wt. %, or, more specifically, 5 wt. % to 20 wt. %”, is inclusive of the endpoints and all intermediate values of the ranges of “0 wt. % to 25 wt. %” and “5 wt. % to 20 wt. %”). The term “combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. The term “or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or elements optionally together with a like or equivalent component or element not listed.

The terms “or/and” and “and/or” mean “either . . . or . . . , or both . . . and . . . ” when referring to two elements, and mean “either . . . , . . . or . . . , or any combination or all thereof” when referring to three or more elements. As an example, the phrase “A or/and B” means “either A or B, or both A and B”, and the phrase “A, B or/and C” means “either A, B or C, or any combination or all thereof”.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values

The term “exemplary” as used herein means “serving as an example, instance or illustration”. Any embodiment or feature characterized herein as “exemplary” is not necessarily to be construed as preferred or advantageous over other embodiments or features.

By “active agent” is meant a compound (including a compound disclosed herein), element, or mixture that when administered to a patient, alone or in combination with another compound, element, or mixture, confers, directly or indirectly, a physiological effect on the subject. The indirect physiological effect may occur via a metabolite or other indirect mechanism. The “active agent” may also potentiate or make more active another active agent. For example, a CB1R antagonist or mGluR5 antagonist may potentiate the activity of another active agent when given in combination with another active agent, for example, by lowering the effective dose of the other active agent.

A “pharmaceutical composition” is a composition comprising at least one active agent, such as a CB1R antagonist or/and a mGluR5 antagonist, or a pharmaceutically acceptable salt, hydrate or solvate thereof, and at least one pharmaceutically acceptable excipient or carrier. A “carrier” is a vehicle or diluent, such as an aqueous or/and non-aqueous solvent system, with which an active agent is provided. A “pharmaceutically acceptable” excipient or carrier is generally safe, non-toxic and neither biologically nor otherwise undesirable, and is acceptable for veterinary use as well as human pharmaceutical use. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents {e.g., saline, buffered saline (e.g., phosphate-buffered saline [PBS]) and isotonic solutions (e.g., Ringer's solution)}, and organic solvents (e.g., dimethyl sulfoxide [DMSO] and alcohols [e.g., ethanol, glycerol and propylene glycol]). Except insofar as any conventional excipient or carrier is incompatible with the active agent (for purposes of the content of a pharmaceutical composition, the term “active agent” or the like encompasses a prodrug), the disclosure encompasses the use of conventional excipients and carriers in formulations containing one or more active agents such as a CB1R antagonist or/and a mGluR5 antagonist. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004). Pharmaceutical compositions meet the U.S. FDA's GMP (good manufacturing practice) standards for human or non-human drugs.

The term “pharmaceutically acceptable” means that a substance is generally safe and non-toxic and does not produce any excessive adverse, allergic or other untoward reactions when administered to an animal such as a human.

A “pharmaceutically acceptable salt” includes a non-toxic acid-addition or base-addition salt of a parent compound, and also includes a pharmaceutically acceptable hydrate or solvate of such a salt. Examples of pharmaceutically acceptable salts include, but are not limited to, mineral acid or organic acid salts of basic atoms or groups such as amines; metal (e.g., alkali metal or alkaline earth metal) or organic (e.g., organic amine) salts of acidic groups such as carboxylic acids; and the like. Pharmaceutically acceptable salts include conventional non-toxic salts and quaternary ammonium salts of the parent compound formed, for example, from non-toxic inorganic or organic acids. For example, conventional non-toxic acid-addition salts include those derived from inorganic acids such as hydrochloric acid, hydrobromic acid, sulfuric acid, sulfamic acid, phosphoric acid, nitric acid and the like; and salts prepared from organic acids such as acetic acid, propionic acid, succinic acid, glycolic acid, stearic acid, lactic acid, malic acid, tartaric acid, citric acid, ascorbic acid, pamoic acid, maleic acid, hydroxylmaleic acid, phenylacetic acid, glutamic acid, benzoic acid, salicylic acid, mesylic acid, esylic acid, besylic acid, sulfanilic acid, 2-acetoxybenzoic acid, fumaric acid, toluenesulfonic acid, methanesulfonic acid, ethane disulfonic acid, oxalic acid, isethionic acid, HOOC—(CH₂)ₙ—COOH where n is 0-4, and the like. Examples of metals useful as cations include without limitation alkali metals (e.g., lithium, sodium, potassium and cesium), alkaline earth metals (e.g., magnesium, calcium and barium), aluminum and zinc. Metal cations can be provided by way of, e.g., inorganic bases, such as hydroxides, carbonates and hydrogen carbonates. Non-limiting examples of organic amines useful for forming base-addition salts include chloroprocaine, choline, cyclohexylamine, dibenzylamine, N,N′-dibenzylethylenediamine, dicyclohexylamine, diethanolamine, ethylenediamine, N-ethylpiperidine, histidine, isopropylamine, N-methylglucamine, procaine, pyrazine, triethylamine, trimethylamine and tromethamine. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editors, Wiley-VCH, 2002.

The terms “treat”, “treating”, and “treatment” include alleviating, ameliorating, reversing or abrogating a medical condition or one or more symptoms or complications associated with the condition, and alleviating, ameliorating or eradicating one or more causes of the condition. Reference to “treatment” of a medical condition includes preventing, precluding, reducing the risk or likelihood of developing, delaying the onset of, reducing the incidence, frequency or severity of, and slowing or stopping the progression of, the condition or one or more symptoms or complications associated with the condition.

In some embodiments, “treatment” or “treating” means providing an active agent to a subject in an amount effective to measurably reduce a central nervous system disorder symptom, slow progression of the central nervous system disorder, or minimize the risk of developing the central nervous system disorder symptom. In an aspect, treatment of the central nervous system disorder symptom may be initiated before the subject presents symptoms of the disease.

The term “dosing regimen” refers to the dosage and frequency of administration, and optionally the length of treatment and route of administration, of a therapeutic agent. The term “treatment regimen” may refer to a dosing regimen depending on the context.

The term “therapeutically effective amount” refers to an amount of a substance that, when administered to a subject, is sufficient to prevent, reduce the risk of developing, delay the onset of, or slow the progression of the medical condition being treated; to alleviate or ameliorate to some extent one or more symptoms or complications of the medical condition; or to treat the medical condition as defined herein. The term “therapeutically effective amount” also refers to an amount of a substance that is sufficient to elicit the biological or medical response of a cell, tissue, organ, system, animal or human which is sought by a researcher, veterinarian, medical doctor or clinician.

In some embodiments, a “therapeutically effective amount” of a CB1R antagonist or a mGluR5 antagonist is an amount effective, when administered to a patient, to provide a therapeutic benefit, such as reduction in the number of fibroblasts at a fibrotic lesion, amelioration or reduction of one or more symptoms of a fibrotic disorder, or improvement in one or more pulmonary function parameters such as pressure-volume loop, tissue stiffness, peripheral airway resistance, forced vital capacity, air flow, inspiratory capacity, and inhaled air amount. Thus, a therapeutically effective amount of a compound is also an amount sufficient to significantly reduce the indicia of the disease or condition being treated. A significant reduction is statistically significant in a standard parametric test of statistical significance, such as Student's t-test, in which p<0.05.

The term “medical conditions” (or “conditions” for brevity) includes diseases and disorders. The terms “diseases” and “disorders” are used interchangeably herein.

“Administering” means giving, providing, applying, or dispensing by any suitable route. Administration of a combination of active agents includes administration of the combination in a single formulation or unit dosage form, administration of the individual active agents of the combination concurrently but separately, or administration of the individual active agents of the combination sequentially by any suitable route. The dosage of the individual active agents of the combination may require more frequent administration of one of the active agent(s) as compared to the other active agent(s) in the combination. Therefore, to permit appropriate dosing, packaged pharmaceutical products may contain one or more dosage forms that contain the combination of active agents, and one or more dosage forms that contain one of the combination of active agents, but not the other active agent(s) of the combination.

The term “combination therapy” refers to the administration of two or more therapeutic (active) agents to treat a medical condition or disorder. Such administration encompasses co-administration of the therapeutic agents in a substantially simultaneous manner, such as in a single dosage form having a fixed ratio of active ingredients or in separate dosage forms for each active ingredient. In addition, such administration encompasses administration of each therapeutic agent in a sequential manner, either at approximately the same time or at different times. In either case, the treatment regimen provides the beneficial effects of each therapeutic agent in the drug combination in treating the condition or disorder.

A “patient” or a “subject” is a member of any mammalian or non-mammalian species which may be in need of medical treatment. Medical treatment can include treatment of, e.g., an incipient or existing condition, or diagnostic treatment. Mammals include without limitation primates (e.g., humans), canines, felines, ungulates (e.g., bovines, equines, ovine and swine [e.g., pigs]), rodents and lagomorphs. In some embodiments, the subject or patient is a human or a non-human animal having commercial importance (e.g., livestock or a domesticated animal). In certain embodiments, the patient is a human patient.

A significant change or difference is any detectable change or difference that is statistically significant in a standard parametric test of statistical significance such as Student's T-test, where p<0.05.

### Treatment of Fibrotic Disorders by Reducing the Level or Activity of mGluR5 or/and CB1R

In some embodiments, the invention is directed to methods of treating a fibrosing disorder in a mammal comprising decreasing the activity of CB1R or mGluR5 or a combination thereof, in the fibrocytes and/or fibroblasts present at or associated with a fibrotic lesion in said fibrosing disorder.

In one aspect, decreasing the activity of CB1R or mGluR5 is by reducing the expression of the gene encoding CB1R or mGluR5. Gene expression can be reduced at the transcription stage by reducing CNR1 (CB1R) or GRM5 (mGluR5) mRNA, for example, by introducing a small interfering RNA (siRNA) molecule targeting CB1R or mGluR5 mRNA, or at the translation stage, by inhibiting translation of CB1R or mGluR5 mRNA, for example, by using microRNAs designed to bind CB1R or mGluR5 mRNA. Optionally, the expression of one or more genes associated with the activity of CB1R or mGluR5 is decreased. For example, the expression of at least one effector or activator gene associated with CB1R or mGluR5 activity is decreased. In another aspect, CB1R or mGluR5 activity is reduced by reducing the signaling of the receptors in the lungs by delivering one or more active agents to the lungs locally via aerosol, nebulizer, or inhaler.

In yet another aspect, decreasing the activity of CB1R or mGluR5 comprises administering to a mammal such as a human an agent, such as a ligand or drug, that blocks or dampens a biological response by binding to and blocking the receptor in an amount effective to treat, such as alleviate one or more symptoms, of a fibrosing disorder. Such drugs may be pharmacological drugs known to be antagonists of either CB1R or mGluR5. Examples of drugs known as CB1R antagonists include, but are not limited to, peripheral CB1R antagonists, for example, zevaquenabant (MRI-1867), MRI-1891 and TM-38837. Examples of drugs known as mGluR5 antagonists include, but are not limited to, negative allosteric modulators of mGluR5 glutamate signaling such as fenobam, basimglurant, raseglurant and dipraglurant, and selective mGluR5 antagonists that inhibit activation of the mGluR5receptor such as CTEP (2-chloro-4-((2,5-dimethyl-1-(4-(trifluoromethoxy)phenyl)-1H-imidazol-4-yl)ethynyl)pyridine), mavoglurant, auglurant, and remeglurant. In one aspect, the negative allosteric modulator of mGluR5 is fenobam.

In one aspect, decreasing the activity of mGluR5 comprises administering to a subject a mGluR5 antagonist. In one aspect, the mGluR5 antagonist is a negative allosteric modulator of mGluR5. In another aspect, the mGluR5 antagonist is a selective inhibitor of mGluR5 activation. In another aspect, the mGluR5 antagonist comprises a combination of a selective inhibitor of mGluR5 activation and a negative allosteric modulator of mGluR5. In one aspect, the negative allosteric modulator of mGluR5 is any of, or a combination of, fenobam, basimglurant, raseglurant, and diplaglurant. In some aspects, the selective inhibitor of mGluR5 activation is any of, or a combination of, CTEP, mavoglurant, auglurant, and remeglurant.

In another aspect, a fibrotic disorder is treated with a combination of a mGluR5 antagonist and a CB1R antagonist (e.g., a peripheral CB1R antagonist). Such a combination can provide greater efficacy compared to treatment with either therapeutic agent alone, by reducing the effects/activity of CB1R and the effects/activity of mGluR5. In some embodiments, the combination of a mGluR5 antagonist and a CB1R antagonist (e.g., a peripheral CB1R antagonist) has a synergistic effect in the treatment of a fibrotic disorder.

In one aspect, treatment of a fibrotic disorder in a subject comprises administering to the subject a combination therapy comprising a mGluR5 antagonist and a CB1R antagonist (e.g., a peripheral CB1R antagonist). In one aspect, the treatment comprises administering a peripheral CB1R antagonist and a negative allosteric modulator of mGluR5. In another aspect, the treatment comprises administering a peripheral CB1R antagonist and a selective inhibitor of mGluR5 activation. In yet another aspect, the treatment comprises administering a peripheral CB1R antagonist, a negative allosteric modulator of mGluR5, and a selective inhibitor of mGluR5 activation. In some aspects, the peripheral CB1R antagonist is zevaquenabant and the negative allosteric modulator of mGluR5 is fenobam. In some aspects, the peripheral CB1R antagonist is zevaquenabant and the negative allosteric modulator of mGluR5 is basimglurant. In some aspects, the peripheral CB1R antagonist is zevaquenabant and the negative allosteric modulator of mGluR5 is raseglurant. In some aspects, the peripheral CB1R antagonist is zevaquenabant and the negative allosteric modulator of mGluR5 is dipraglurant. In some aspects, the peripheral CB1R antagonist is zevaquenabant and the selective inhibitor of mGluR5 activation is CTEP. In some aspects, the peripheral CB1R antagonist is zevaquenabant and the selective inhibitor of mGluR5 activation is mavoglurant. In some aspects, the peripheral CB1R antagonist is zevaquenabant and the selective inhibitor of mGluR5 activation auglurant. In some aspects, the peripheral CB1R antagonist is zevaquenabant and the selective inhibitor of mGluR5 activation is remeglurant.

In some aspects, treatment of a fibrotic disorder in a subject using a mGluR5 antagonist or/and a CB1R antagonist (e.g., a peripheral CB1R antagonist) comprises administering to the subject a maximally effective dose of the antagonist(s). The maximally effective dose is generally defined as the range between the minimum effective dose (MED) and the maximum tolerated dose (MTD). The MED is defined as the lowest dose level of a pharmaceutical product that provides a clinically significant response in average efficacy, which is also statistically significantly superior to the response provided by a placebo. Similarly, the MTD is the highest possible but still tolerable dose level with respect to a pre-specified clinical limiting toxicity. For example, in one aspect, treatment of a fibrotic disorder in a subject comprises administering to the subject a daily dose of a peripheral CB1R antagonist such as zevaquenabant at about 1-200 mg orally or MRI-1891 at about 0.5-100 mg, or/and a daily dose of a mGluR5 antagonist such as CTEP at about 0.5-100 mg/kg orally, fenobam at about 50-700 mg orally, basimglurant at about 0.5-5 mg orally, dipraglurant at about 25-150 mg orally, mavoglurant at about 25-200 mg orally, or auglurant at about 25-200 mg orally. The daily dose of a peripheral CB1R antagonist or/and a mGluR5 antagonist can be taken in a single dose or in divided doses (e.g., twice or thrice a day to reach the total daily dose).

In some embodiments, the invention provides a pharmaceutical composition for treating a fibrotic disease comprising a mGluR5 antagonist. In another embodiment, the invention provides a pharmaceutical composition for treating a fibrotic disease comprising a CB1R antagonist such as a peripheral CB1R antagonist. In yet another embodiment, the invention provides a pharmaceutical composition for treating a fibrotic disease comprising a mGluR5 antagonist in combination with a CB1R antagonist such as a peripheral CB1R antagonist.

Other embodiments provide for the use of a mGluR5 antagonist or/and a CB1R antagonist (e.g., a peripheral CB1R antagonist) in the manufacture of a pharmaceutical composition for treating a fibrotic disease in a subject and the use of a mGluR5 antagonist or a CB1R antagonist (e.g., a peripheral CB1R antagonist) in the manufacture of a pharmaceutical composition for treating a fibrotic disease in a subject when administered in combination with a CB1R antagonist (e.g., a peripheral CB1R antagonist) or a mGluR5 antagonist, respectively.

In some embodiments, the pharmaceutical composition comprising a mGluR5 antagonist or/and a CB1R antagonist (e.g., a peripheral CB1R antagonist) is provided in a kit, and the kit further comprises a package insert comprising instructions for treating a fibrotic disease in a subject with a mGluR5 antagonist or a CB1R antagonist (e.g., a peripheral CB1R antagonist), or a combination of a mGluR5 antagonist and a CB1R antagonist (e.g., a peripheral CB1R antagonist).

In additional embodiments, treatment of a fibrotic disorder in a subject comprises administering to the subject a mGluR5 antagonist or/and a CB1R antagonist (e.g., a peripheral CB1R antagonist) in combination with one or more other therapeutic agents. In some embodiments, the one or more other therapeutic agents comprise one or more therapeutic agents used to treat a fibrotic disorder, such as an anti-inflammatory agent or an immunosuppressant (e.g., azathioprine or a corticosteroid such as prednisone), or pirfenidone or nintedanib for treatment of IPF or other fibrotic disorder. In further embodiments, the one or more other therapeutic agents comprise an inhibitor of transforming growth factor beta (TGF-β), the main promoter of fibrosis, or an antagonist of a TGF-β receptor.

The fibrotic diseases and fibrosing disorders treatable by the methods, therapeutic agents and pharmaceutical compositions described herein include, but are not limited to, pulmonary fibrosis, chronic obstructive pulmonary disease (COPD), idiopathic pulmonary fibrosis (IPF), Hermansky-Pudlak syndrome pulmonary fibrosis (HPSPF), interstitial lung disease (including usual interstitial pneumonia [UIP] and scleroderma-related interstitial lung disease [Ssc-ILD]), respiratory bronchiolitis/interstitial lung disease, hypersensitivity pneumonitis, primary pulmonary hypertension (including prevention of the formation of plexiform lesion), chronic graft versus host disease (cGVHD), hepatic/liver fibrosis, cirrhosis, non-alcoholic steatohepatitis (NASH), cardiac fibrosis, myocardial fibrosis (e.g., interstitial fibrosis, subepicardial fibrosis, subendocardial fibrosis and replacement fibrosis), cardiomyopathy, congestive heart failure, renal fibrosis, chronic renal disease, diabetic nephropathy, retroperitoneal fibrosis (Ormond's disease), nephrogenic systemic fibrosis, Schistosoma mansoni infection, herpes virus-associated diseases (including lung and dermatological manifestations), SARS-CoV-2- and variants thereof-related pulmonary diseases, alcohol use disorder (AUD)-related lung injury, acute respiratory distress syndrome (ARDS), synthetic cannabinoids-induced lung injury and respiratory failure, keloid scarring, lupus, nephrogenic fibrosing dermopathy, fibrosing lesions associated with Schistosoma japonicum infection, autoimmune diseases (e.g., rheumatoid arthritis), pathogenic fibrosis, Lyme disease, stromal remodeling in pancreatitis, stromal fibrosis, uterine fibroids, ovarian fibrosis, corneal fibrosis, ischemia-related conditions including pre-ischemic and post-ischemic conditions (e.g., congestive heart failure), post-surgical scarring (including abdominal adhesions), wide angle glaucoma trabeculotomy, and any combinations thereof. In certain embodiments, the fibrosing disorder is chronic pulmonary fibrosis.

Pulmonary fibrosis can occur in patients with a variety of disorders, such as congestive heart failure, atypical pneumonia (including Pneumocystis pneumonia) and lymphangitic spread of cancer. Environmental or occupational exposures, including inhalational exposures to inorganic dusts, e.g., silicone, asbestos, beryllios, and black lung have also been recognized as causing lung diseases characterized by pulmonary fibrosis. Pulmonary fibrosis may also develop from exposure to protein antigens (e.g., farmer's lung, pigeon-breeder's lung, hot-tub lung) and exposure to toxic gases, fumes, aerosols, and vapors (e.g., silo-filler's disease). Exposure to radiation, including ionizing radiation used in medical applications, is also a well-recognized cause of pulmonary fibrosis. Pulmonary fibrosis may also occur in rheumatologic or connective-tissue diseases, such as scleroderma, rheumatoid arthritis, mixed connective-tissue disease, and systemic lupus erythematosus. Moreover, pulmonary fibrosis may occur in pulmonary-renal syndromes (e.g., Wegner and Goodpasture diseases), sarcoidosis and other granulomatous diseases (e.g., berylliosis), systemic disorders such as hepatitis C, inflammatory bowel disease, acquired immunodeficiency syndrome, and idiopathic or rare diffuse parenchymal lung diseases (DPLDs), such as cryptogenic organizing pneumonia (COP, idiopathic), pulmonary Langerhans cell histiocytosis (rare), and eosinophilic pneumonia. In addition, pulmonary fibrosis may occur in tuberous sclerosis, neurofibromatosis, Niemann-Pick disease, Gaucher disease, and Hermansky-Pudlak syndrome.

Pulmonary fibrosis can result in pulmonary hypertension due to the scarred tissue affecting the pulmonary arteries by compressing the vessels, leading to increased pressure in the pulmonary arteries and the right heart ventricle, which in turn increases left ventricular pressure. Therefore, in another aspect, the disclosure provides for treatment of pulmonary hypertension using a mGluR5 antagonist or a CB1R antagonist (e.g., a peripheral CB1R antagonist), or a combination thereof. Pulmonary hypertension (PH) and PH-associated disorders include without limitation functional classes I to IV pulmonary hypertension, primary pulmonary hypertension (PPH), secondary pulmonary hypertension (SPH), familial PPH, sporadic PPH, precapillary pulmonary hypertension, pulmonary arterial hypertension (PAH), pulmonary venous hypertension, idiopathic pulmonary hypertension, thrombotic pulmonary arteriopathy (TPA), plexogenic pulmonary arteriopathy, and pulmonary hypertension associated with, related to, or secondary to left ventricular dysfunction, mitral valvular disease, constrictive pericarditis, aortic stenosis, cardiomyopathy, mediastinal fibrosis, anomalous pulmonary venous drainage, pulmonary veno-occlusive disease, collagen vascular disease, congenital heart disease, HIV virus infection, exposure to drugs and toxins such as fenfluramines, chronic obstructive pulmonary disease, interstitial lung disease, a sleep disorder or breathing affected thereby, alveolar hypoventilation disorder, chronic exposure to high altitude, neonatal lung disease, alveolar-capillary dysplasia, sickle cell disease or other coagulation disorder such as chronic thromboemboli, connective tissue disease, lupus, schistosomiasis, sarcoidosis or pulmonary capillary hemangiomatosis.

In some embodiments, pulmonary hypertension treated with a mGluR5 antagonist or/and a CB1R antagonist (e.g., a peripheral CB1R antagonist) is pulmonary hypertension associated with disorders of the respiratory system and/or hypoxemia, including chronic obstructive pulmonary disease, interstitial lung disease, sleep disorders and breathing affected thereby, alveolar hypoventilation disorders, chronic exposure to high altitude, neonatal lung disease and alveolar-capillary dysplasia. In certain embodiments, the pulmonary hypertension is associated with chronic obstructive pulmonary disease.

In some embodiments, a mGluR5 antagonist or/and a CB1R antagonist (e.g., a peripheral CB1R antagonist) is/are used in combination with one or more other therapeutic agents to treat pulmonary hypertension. In some embodiments, the one or more other therapeutic agents are selected from anticoagulants, diuretics, cardiac glycosides, calcium channel blockers, vasodilators, prostacyclin analogs, endothelin receptor (e.g., ETA or/and ETB2) antagonists, phosphodiesterase (e.g., PDE5) inhibitors, beta-2 agonists, antimuscarinics, endopeptidase inhibitors, lipid-lowering agents, and thromboxane inhibitors, and combinations thereof.

In additional embodiments, an agent that decreases the level or activity of mGluR5 (e.g., a mGluR5 antagonist) or/and an agent that decreases the level or activity of CB1R (e.g., a CB1R antagonist such as a peripheral CB1R antagonist) is/are used to treat fibrosis that is associated with, induced by or caused in response to various cancer treatments such as radiation therapy. In some embodiments, such agent(s) is/are used in conjunction with radiation therapy in the treatment of a tumor or cancer. Radiation-induced pulmonary laminitis and subsequent pulmonary fibrosis are side effects of radiation therapy that hamper the efficacy of the radiation therapy. In the treatment of cancer, radiation therapy is typically administered at a dose of 20-85 gray. However, patient studies have shown an almost linear relationship of lung toxicity in the form of pulmonary laminitis as the radiation dose increases. Subsequently, fibrotic lesions are also seen. In some embodiments, an agent that decreases the level or activity of CB1R and/or an agent that decreases the level or activity of mGluR5, such as in fibrocytes, fibroblasts or/and macrophages in the pulmonary tissues, is/are used to treat radiation-induced pulmonary laminitis and/or radiation-induced pulmonary fibrosis. An agent that decreases the level or activity of mGluR5 (e.g., a mGluR5 antagonist) and/or an agent that decreases the level or activity of CB1R (e.g., a CB1R antagonist such as a peripheral CB1R antagonist) may be administered before, after and/or concurrently with the radiation therapy. In some embodiments, such agent(s) is/are administered within 1, 2, 3, 4, or 5 days before or/and after the administration of the radiotherapy.

Another cancer therapy that has been shown to lead to significant fibrosis including pulmonary fibrosis is treatment with bleomycin sulfate. Bleomycin is deposited in the skin and lungs, which leads to fibrosis. While cessation of drug administration and administration of corticosteroids are recommended, there is no demonstrated treatment for bleomycin-induced lung injury. In some embodiments, an agent that decreases the level or activity of mGluR5 (e.g., a mGluR5 antagonist) and/or an agent that decreases the level or activity of CB1R (e.g., a CB1R antagonist such as a peripheral CB1R antagonist) is/are administered before, after or concurrently with, or any combination or all thereof, administration of bleomycin sulfate to treat bleomycin-induced fibrosis such as pulmonary fibrosis.

In addition to bleomycin sulfate, other drugs also induce fibrosis and pulmonary diseases, including pulmonary fibrosis. It is known that fibrosis (e.g., pulmonary fibrosis) can be caused by numerous drugs, including chemotherapeutic agents (e.g., bleomycin, busulfan, methotrexate and nitrosoureas), antibiotics (e.g., nitrofurantoin), antiarrhythmics (e.g., amiodarone and tocainide), anti-inflammatory medications (e.g., sulfasalazine), gold, penicillamine, and illicit drugs (e.g., crack cocaine and heroin). For example, up to 25% of cancer patients receiving a nitrosourea chemotherapeutic agent (e.g., carmustine, lomustine or semustine, especially carmustine) develop early-onset pulmonary fibrosis within 36 months of initiation of nitrosourea therapy. In some embodiments, an agent that decreases the level or activity of mGluR5 (e.g., a mGluR5 antagonist) or/and an agent that decreases the level or activity of CB1R (e.g., a CB1R antagonist such as a peripheral CB1R antagonist), such as in fibrocytes or/and fibroblasts in the pulmonary tissues, is/are used in the treatment of drug- or substance-induced fibrosis (e.g., pulmonary fibrosis). Drugs and substances that can induce fibrosis (e.g., pulmonary fibrosis) include without limitation amphotericin B, bleomycin, bromocriptine, busulfan, carbamazepine, chlorambucil, cocaine, cyclophosphamide, diphenylhydantoin, ergotamine, flecainide, heroin, melphalan, methadone, methotrexate, methylphenidate, methysergide, mineral oil, nitrofurantoin, nitrosoureas, procarbazine, silicone, sulfasalazine, tocainide, the vinca alkaloid class of agents (e.g., vinblastine and vincristine), mitomycin and antimicrobial agents. The anti-mGluR5 agent or/and the anti-CB1R agent can be administered prior to, concurrently with or/and after administration of or exposure to the fibrosis-inducing drug or substance. In some embodiments, an anti-mGluR5 agent or/and an anti-CB1R agent is/are used in combination with an anti-inflammatory agent or an immunosuppressant (e.g., azathioprine or a corticosteroid such as prednisone) to treat a drug- or substance-induced fibrosis (e.g., pulmonary fibrosis).

In some embodiments, one or more therapeutic agents described herein are administered locally to the site of a fibrosing lesion. In more specific embodiments, the fibrosing lesion is in a lung and the one or more therapeutic agents are contacted locally with said lesion. In certain embodiments, the pharmaceutical composition (e.g., liposomes or polymer nanoparticles) containing the one or more therapeutic agents comprises a targeting moiety to specifically locate said agent(s) to the site of a fibrosing lesion. In some embodiments, the one or more therapeutic agents are administered into the lungs by oral inhalation using an inhaler, aerosol, or nebulizer.

In addition, the invention provides the use of an agent that decreases the level or activity of mGluR5 (e.g., a mGluR5 antagonist) and/or an agent that decreases the level or activity of CB1R (e.g., a CB1R antagonist such as a peripheral CB1R antagonist) in fibrocytes and/or fibroblasts at a fibrotic lesion in the preparation of a medicament for the treatment of a fibrosing disorder.

The therapeutic agents described herein, including mGluR5 antagonists and CB1R antagonists, can be administered by any suitable route for the treatment of fibrotic disorders. In some embodiments, the therapeutic agents described herein, including mGluR5 antagonists and CB1R antagonists, are administered orally. In other embodiments, the therapeutic agents described herein, including mGluR5 antagonists and CB1R antagonists, are administered by topical (e.g., dermal, transdermal, mucosal, transmucosal or intranasal) administration, injection, oral or nasal inhalation into the lungs, continuous release by depot or pump, or any combinations thereof. Other conventional routes of administration, such as parenteral (e.g., subcutaneous, intravenous, intradermal, intramuscular, intramammary, intraperitoneal or intrathecal), intraocular, retrobulbar, intrapulmonary (e.g., term release), aerosol, sublingual, buccal, nasal, anal, vaginal, or transdermal administration, or by surgical implantation at a particular site, can also be used when oral administration is not suitable. In a combination therapy, the routes of administration may differ for the therapeutic agents, if not provided in the same pharmaceutical composition, such as one therapeutic agent being administered by inhalation and another therapeutic agent being administered orally.

The therapeutic agents described herein, including mGluR5 antagonists and CB1R antagonists, can be administered as neat substances, but are preferably administered as a pharmaceutical composition. Accordingly, the disclosure provides pharmaceutical compositions comprising one or more therapeutic agents described herein, such as a mGluR5 antagonist or/and a CB1R antagonist, or a pharmaceutically acceptable salt, hydrate, solvate, polymorph, isomer, prodrug, etc. thereof, together with at least one pharmaceutically acceptable excipient or carrier.

A pharmaceutical composition containing one or more therapeutic agents described herein, such as a mGluR5 antagonist or/and a CB1R antagonist, may also contain one or more other agents that facilitate delivery of the therapeutic agent(s) to the target tissue(s). As an example, for a transmucosal administration such as administration into the lungs, a pharmaceutical composition can contain a mucolytic agent such as acetylcysteine, trypsin or ambroxol.

The formulation of a pharmaceutical composition depends in part on the selected route of administration. Appropriate salt(s) and buffer(s) can be added to render a pharmaceutical composition stable and allow for uptake of the composition at the target site.

For oral administration, a pharmaceutical composition can be formulated as a tablet, capsule, pill or other oral delivery form. When formulated for oral administration, a pharmaceutical composition may contain from about 0.1 to about 99 weight % (wt.%), from about 25 wt. % to about 50 wt. %, or from about 5 wt. % to about 75 wt. % of a mGluR5 antagonist and/or a CB1R antagonist, or at least about 5 wt. % of a mGluR5 antagonist and/or a CB1R antagonist. For parenteral administration, one or more therapeutic agents can be provided in a sterile aqueous solution containing optionally isotonic agent(s) (e.g., NaCl) and buffering agent(s) (e.g., sodium citrate), optionally in a single-use pre-filled syringe. A therapeutic agent can also be provided in lyophilized form to be reconstituted prior to administration. Buffer(s) and solution(s) for the reconstitution of the therapeutic agent may be provided to produce an aqueous composition.

In certain embodiments, the pharmaceutical composition is in a unit dosage form that contains from about 0.1 mg to about 2000 mg, from about 10 mg to about 1000 mg, from about 100 mg to about 800 mg, or from about 200 mg to about 600 mg of a CB1R antagonist, and/or from about 0.1 mg to about 2000 mg, from about 10 mg to about 1000 mg, from about 100 mg to about 800 mg, or from about 200 mg to about 600 mg of a mGluR5 antagonist. In other embodiments, a unit dosage form contains about 1-500 mg of a CB1R antagonist, or/and about 1-500 mg of a mGluR5 antagonist.

For treatment of a condition described herein (e.g., a fibrotic disorder), in some embodiments the daily dose of a mGluR5 antagonist or a CB1R antagonist is about 0.1-140 mg, 0.5-100 mg or 1-50 mg per kilogram of body weight. In further embodiments, the daily dose of a mGluR5 antagonist or a CB1R antagonist is about 0.5 mg-7 g, 1-500 mg, 25-500 mg, 25-200 mg, 10-100 mg or 100-200 mg. The daily dose can be administered in a single dose or in divided doses (e.g., twice or thrice a day to reach the total daily dose), which may depend on the particular therapeutic agent, the particular route of administration, and the particular disease. The daily dose of a mGluR5 antagonist or a CB1R antagonist can be administered, e.g., as a bolus, via infusion or via a sustained-release composition. Alternatively, a mGluR5 antagonist or a CB1R antagonist may not need to be administered daily, which can be determined by the treating physician.

In some embodiments, treatment with a therapeutic agent described herein, such as a mGluR5 antagonist or a CB1R antagonist, lasts for at least about 2 weeks, one month (4 weeks) or 6 weeks. In further embodiments, treatment with a therapeutic agent described herein, such as a mGluR5 antagonist or a CB1R antagonist, lasts for at least about 3 months, 6 months, 1 year, 2 years or 3 years.

It is understood that the therapeutically effective dose of, the frequency of administration of, and the length of treatment with a particular therapeutic agent may depend on a variety of factors, including the particular disease, the severity of the disease, the potency of the therapeutic agent, the route and time of administration, the age, body weight, general health, gender and diet of the patient, and the response of the patient to the treatment, and can be determined by the treating physician.

An agent that decreases the level or activity of mGluR5 (e.g., a mGluR5 antagonist) or/and an agent that decreases the level or activity of CB1R (e.g., a CB1R antagonist such as a peripheral CB 1R antagonist) can optionally be used in combination with one or more additional therapeutic agents to treat any condition described herein, such as a fibrotic disorder (e.g., pulmonary fibrosis). Fibrosis is initiated when immune cells such as macrophages release soluble factors such as TGF-β that stimulate fibroblasts, which then lay down connective tissue containing collagen, often in response to tissue inflammation or damage. Accordingly, in some embodiments an anti-mGluR5 agent or/and an anti-CB1R agent is/are used in combination with an anti-inflammatory agent or/and an immunosuppressant (e.g., azathioprine or a corticosteroid such as prednisone) to treat a fibrotic disorder (e.g., pulmonary fibrosis). Many immunosuppressants, including the glucocorticoid class of corticosteroids, also possess anti-inflammatory property. The anti-mGluR5 agent or/and the anti-CB1R agent and the additional therapeutic agent(s) can be administered in the same pharmaceutical composition or in separate pharmaceutical compositions, and the administrations of such agents can be concurrent or/and sequential if the latter case.

Non-steroidal anti-inflammatory drugs (NSAIDs) include without limitation:
acetic acid derivatives, such as aceclofenac, bromfenac, diclofenac, etodolac, indomethacin, ketorolac, nabumetone, sulindac, sulindac sulfide, sulindac sulfone and tolmetin;
anthranilic acid derivatives (fenamates), such as flufenamic acid, meclofenamic acid, mefenamic acid and tolfenamic acid;
enolic acid derivatives (oxicams), such as droxicam, isoxicam, lornoxicam, meloxicam, piroxicam and tenoxicam;
propionic acid derivatives, such as fenoprofen, flurbiprofen, ibuprofen, dexibuprofen, ketoprofen, dexketoprofen, loxoprofen, naproxen and oxaprozin;
salicylates, such as diflunisal, salicylic acid, acetylsalicylic acid (aspirin), choline magnesium trisalicylate, and salsalate;
COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-58125, benzothieno[3,2-d]pyrimidin-4-one sulfonamide thio-derivatives, and COX-2 inhibitors derived from Tribulus terrestris;
other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) (e.g., chebulagic acid, licofelone, 2-(3,4,5-trimethoxyphenyl)-4-(N-methylindol-3-yl)thiophene, and di-tert-butylphenol-based compounds [e.g., DTPBHZ, DTPINH, DTPNHZ and DTPSAL]); and
analogs, derivatives and salts thereof.

Immunosuppressants include without limitation interferon-beta (IFN-β), glucocorticoids (infra), antimetabolites (e.g., hydroxyurea [hydroxycarbamide], antifolates [e.g., methotrexate], and purine analogs [e.g., azathioprine, mercaptopurine and thioguanine]), pyrimidine synthesis inhibitors (e.g., leflunomide and teriflunomide), calcineurin inhibitors (e.g., ciclosporin [cyclosporine A], pimecrolimus and tacrolimus), inosine-5′-monophosphate dehydrogenase (IMPDH) inhibitors (e.g., mycophenolic acid and derivatives thereof [e.g., mycophenolate sodium and mycophenolate mofetil]), mechanistic/mammalian target of rapamycin (mTOR) inhibitors (e.g., rapamycin [sirolimus], deforolimus [ridaforolimus], everolimus, temsirolimus, umirolimus [biolimus A9],zotarolimus and RTP-801), modulators of sphingosine-1-phosphate receptors (e.g., S1PR1) (e.g., fingolimod), and serine C-palmitoyltransferase inhibitors (e.g., myriocin), and analogs, derivatives and salts thereof.

The glucocorticoid class of corticosteroids has anti-inflammatory and immunosuppressive properties. Glucocorticoids include without limitation hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), carbonates (e.g., prednicarbate), and analogs, derivatives and salts thereof.

The therapeutic agents described herein, including mGluR5 antagonists and CB1R antagonists, and pharmaceutical compositions containing them, can be provided in a kit. When used as a combination, two or more therapeutic agents such as a mGluR5 antagonist and a CB1R antagonist can be provided in the same pharmaceutical composition or in separate pharmaceutical compositions. The kit includes a package insert containing instructions for administering the therapeutic agent(s) or the pharmaceutical composition(s) to treat any condition described herein, such as a fibrotic disorder. If not provided in oral dosage form(s) such as tablet(s) or capsule(s), the therapeutic agent(s) such as a mGluR5 antagonist or/and a CB1R antagonist can be provided, e.g., in pre-filled syringe(s) or in vial(s) accompanied by syringe(s) and needle(s) for parenteral administration, or in a formulation suitable for oral inhalation, in which case the kit can contain an inhaler (e.g., a metered-dose inhaler, a dry powder inhaler, a soft mist inhaler, a smart inhaler or a nebulizer).

The disclosure is further illustrated by the following non-limiting examples.

### EXAMPLES

### Materials and Methods:

### Chemicals

CTEP, fenobam and basimglurant were obtained from MedChem Express (New Jersey, USA). Pharmaceutical-grade bleomycin was obtained from Hospira (Lake Forest, Illinois, USA). All the other chemicals were obtained from Sigma-Aldrich (St. Louis, Missouri, USA).

### Experimental Drug Treatment

The compounds were administered by oral gavage once daily as indicated. The vehicle was a 1:1:18 ratio of DMSO:Tween® 80:saline. Oral formulations were applied at 0.3 and 3 mg/mL concentrations to achieve doses of 3 and 30 mg/kg, respectively.

### Animals

All animal procedures were conducted in accordance with the rules and regulations of the Institutional Animal Care and Use Committee of the National Institutes of Alcohol Abuse and Alcoholism (NIAAA), under the protocols of LPS-GK1. Thirteen-week-old male C57BL/6J mice were obtained from The Jackson Laboratory (Bar Harbor, Maine, USA). Grm5^{-/-} (mGluR5 KO) mice (JAX 003121) were purchased from The Jackson Laboratory. Cnr14^{-/-} (CB1R KO) mice were generated as described in Zimmer et al., Proc. Natl. Acad. Sci. USA, 96:5780-5785 (1999). Cnr1^{-/-} and Grm5^{-/-} mice were on a C57BL/6J genetic background. Both Grm5^{-/-} and Cnr1^{-/-} mice colonies were maintained with heterozygous breeding. Mice were housed individually under a 12-hour light/dark cycle and fed a standard diet, ad libitum (Teklad NIH-31; Envigo, Huntingdon, UK).

### Oropharyngeal Aspiration of Bleomycin

A bleomycin-induced pulmonary fibrosis model by delivery of bleomycin via oropharyngeal aspiration was generated as described in Park et al., Am. J. Respir. Cell Mol. Biol., 62(2):178-190 (2020). Briefly, bleomycin was delivered to mice anesthetized with ketamine/xylazine through the oropharynx at 1 U/kg dose using a sterile 100 μL pipette during inspiration at a volume of 100 μL/50 g body weight. Sterile saline was used as vehicle and applied to the control groups. The animals were then allowed to recover from the anesthesia.

### Survival Analysis for Mice

Survival curves were plotted using GraphPad Prism 9 software. Survival was analyzed by using Log-rank (Mantel-Cox) test for assessing the statistical difference between animal groups.

### Lung Function Measurements in Pale Ear Mice

Respiratory system mechanics measurements were performed using the FlexiVent FX system (SCIREQ Inc., Montreal, Canada), which is equipped with an FX1 module and negative pressure forced expiration (NPFE) extension for mice. FlexiWare v7.2 software was used to operate the system. Forced oscillation techniques and forced expiration measurements were conducted as described in McGovern et al., J. Vis. Exp., 75:e50172 (2013) and Devos et al., Respir. Res., 18:123 (2017). Lung function measurements were performed at the end of the study as a terminal procedure. Mice were anesthetized by intraperitoneal (i.p.) injection of ketamine/xylazine, then an 18-gauge metal cannula was inserted into the trachea by a small incision. Pancuronium was then administered by i.p. injection (0.8 mg/kg) to induce paralysis before connecting mice to FlexiVent and starting ventilation. Pressure-volume (PV) curve, airway resistance, tissue damping (G), tissue elastance (H), forced expiratory volume per 0.1 seconds (FEV 0.1), forced vital capacity (FVC), inspiratory capacity (IC), and inhaled air amount (A) parameters were measured. Mouse tissue was collected after performing lung function tests.

### Hydroxyproline (Hyp) Measurement

The degree of lung fibrosis was quantified biochemically by measuring the hydroxyproline content of lung extracts using liquid chromatography/tandem mass spectrometry (LC-MS/MS) as described in Park et al., Am. J. Respir. Cell Mol. Biol., 62(2):178-190 (2020).

### Measurement of Glutamate (L-Glutamic Acid) Level in Lungs by LC-MS/MS.

The lung tissue was homogenized in 600 μL of ice-cold 0.1 N perchloric acid (PCA) containing 2% EDTA and 1% ethanol using a Precellys tissue homogenizer. Homogenized samples were vortexed and centrifuged at 10,000 g for 10 min, and 10 μL hydrolysate was diluted 100-fold by the addition of 990 μL of 0.1 N PCA. LC-MS/MS analyses were conducted on an Agilent 6470 triple quadrupole mass spectrometer coupled to an Agilent 1260 LC system. Glutamate was separated using an Intrada Amino Acid column (50 mm×3 mm column, 3 μm; Imtakt) at 40° C. Mobile phase A contained 9/75/16/0.3 acetonitrile/tetrahydrofuran/25 mM ammonium formate/formic acid (vol/vol/vol/vol). Mobile phase B contained 20/80 acetonitrile/100 mM ammonium formate (vol/vol). Gradient elution (600 μL min⁻¹) was initiated and held at 0% B for 3 min, followed by a linear increase to 17% B by 6.5 min. A step was increased to 100% B and held until 10 min, followed by a linear decrease to 0% B by 11 min and held until 13 min. The mass spectrometer was set for Agilent jet stream ionization source and operated in positive-ion mode. The source parameters were capillary voltage, 3,500V; gas temperature, 300° C.; sheath gas temperature, 25°° C., sheath gas flow, 10 L min⁻¹, gas flow, 5 L min⁻¹; nitrogen was used as the nebulizing gas. Collision-induced dissociation (CID) was conducted using nitrogen. Glutamate level was analyzed by multiple-reaction monitoring. The molecular ion and fragments for glutamate were measured as follows: m/z 148.1→77.2 and 148.1→39.2 (CID energy of 40V and 80V, respectively). Levels of glutamate were determined against a standard curve, using L-glutamic acid as standard (Sigma). Values are expressed as nmol mg⁻¹ wet tissue.

### Real-Time PCR Analyses

RNA extraction was performed using RNeasy Mini Kits from Qiagen (Valencia, California, USA). One microgram of total RNA was reverse-transcribed to cDNA using Bio-Rad iScript cDNA synthesis kit (Hercules, California). Expression of the target gene was quantified with gene-specific primers and PowerSYBRGreen master mix using a QuantStudio 3 Real-Time PCR instrument from Applied Biosystems. Pre-designed mouse Tbp (QT00198443), Grm5 (QT00288596). The house-keeping gene TATA-Box Binding Protein (Tbp) was used as a loading control. Gene expression values were calculated based on the ΔΔCt method.

### Transcriptomics Analysis

RNA was isolated from the control and fibrotic lung tissues of wt, mGluR5 KO, and CB1R KO mice. Then, RNA sequencing was conducted by Azenta Genewiz (New Jersey, USA) using Poly-A library selection on Illumina NovaSeq6000. Raw RNA-sequencing results were quantified using Kallisto [Bray et al., Nat. Biotechnol., 34(5):525-527 (2016)] with the Ensembl mouse reference genome (GRCm39) [Zerbino et al., Nucleic Acids Res., 46:D754-D761 (2018)]. The quantification data was used for differential expression analysis using DeSeq2 [Love et al., Genome Biol., 15:550 (2014)] and generation of co-expression network using a previously described method [Arif et al., eLife, 10:e66921 (2021)] using top 10% correlation results.

### Example 1. Effects of Knockout and Antagonism of mGluR5 in a Bleomycin-Induced Pulmonary Fibrosis Mouse Model

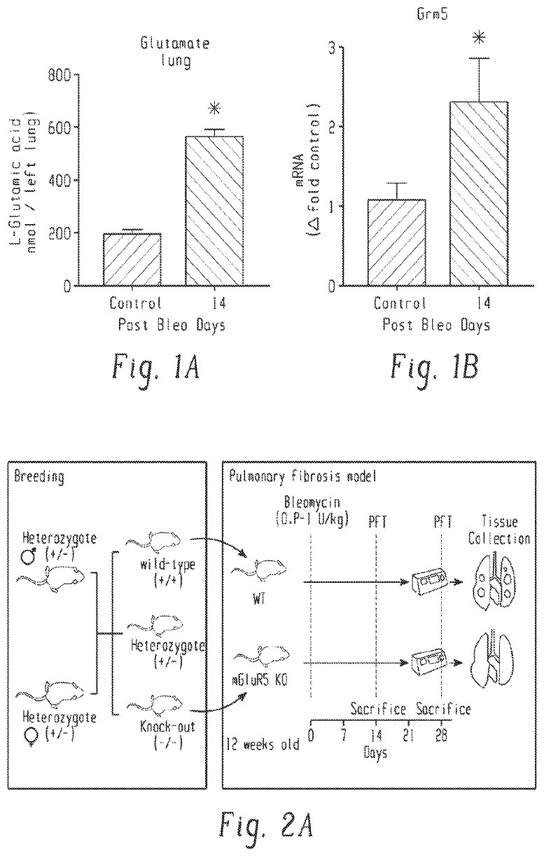

As described in Park et al., Am. J. Respir. Cell Mol. Biol., 62(2):178-190 (2020), mice manifested significant pulmonary fibrosis 14 days after a single oropharyngeal bleomycin (1 U/kg) instillation. Accordingly, the 14 day post-bleomycin timepoint was used to study fibrotic lung disease in mice. As seen in human idiopathic pulmonary fibrosis (IPF) [Zhao et al., BMJ Open Respir. Res., 4:e000183 (2017)], glutamate level significantly increased in fibrotic lungs in mice (FIG. 1A). It was recently shown that alcohol-induced increase in glutamate level in the liver contributes to the pathology of alcoholic liver disease (ALD) through activation of metabotropic glutamate receptor 5 (mGluR5) [Choi et al., Cell Metab., 30:877-889 (2019)]. To investigate expression levels of Grm5, which encodes mGluR5, in pulmonary fibrosis, quantitative PCR (qPCR) was performed. mGluR5 expression significantly increased in fibrotic lungs in mice (FIG. 1B). Since significant increases in both glutamate level and mGluR5 gene expression occurred, the potential pathologic role of mGluR5 in pulmonary fibrosis was explored with a loss of function approach using mGluR5 knockout (KO) mice and wild-type (wt) littermate in the bleomycin-induced pulmonary fibrosis model.

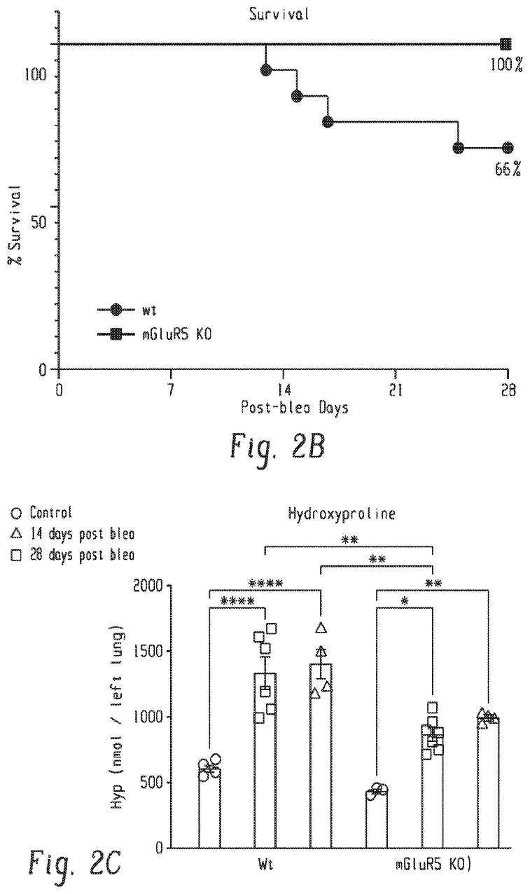

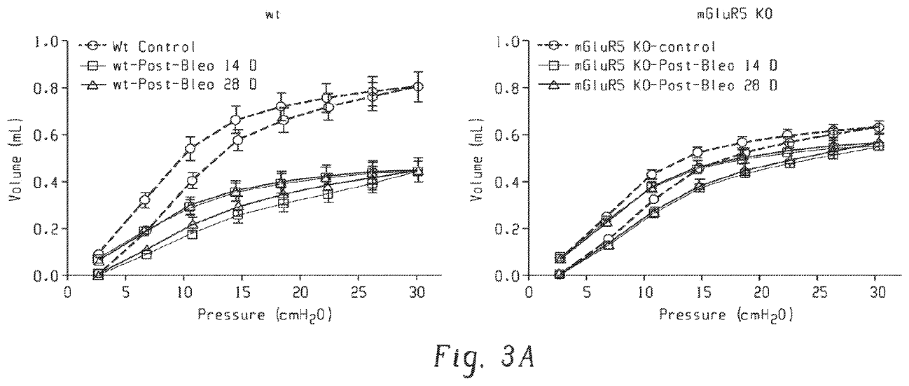

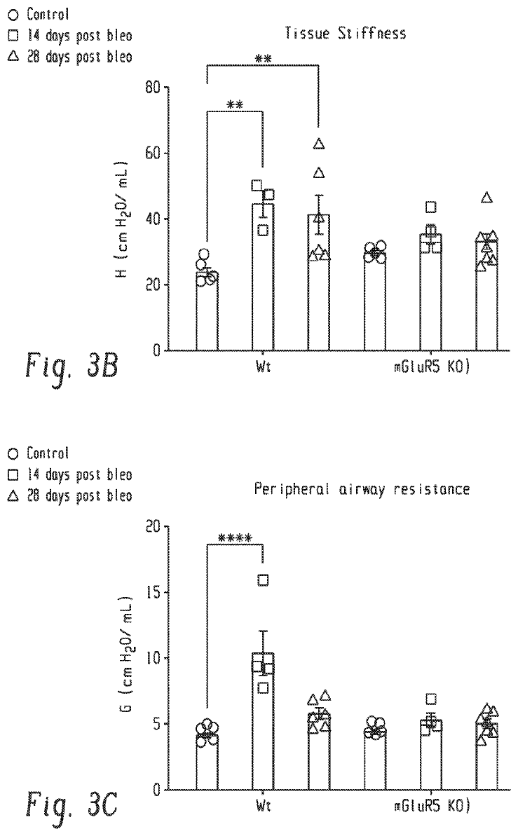

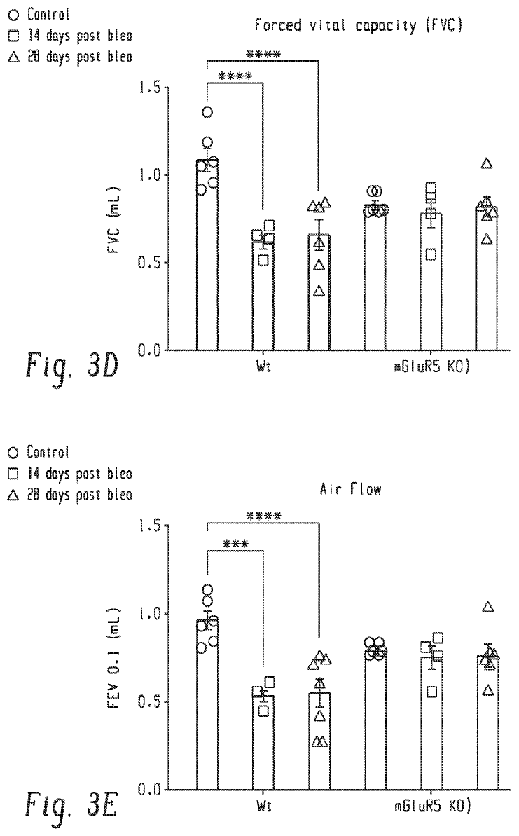

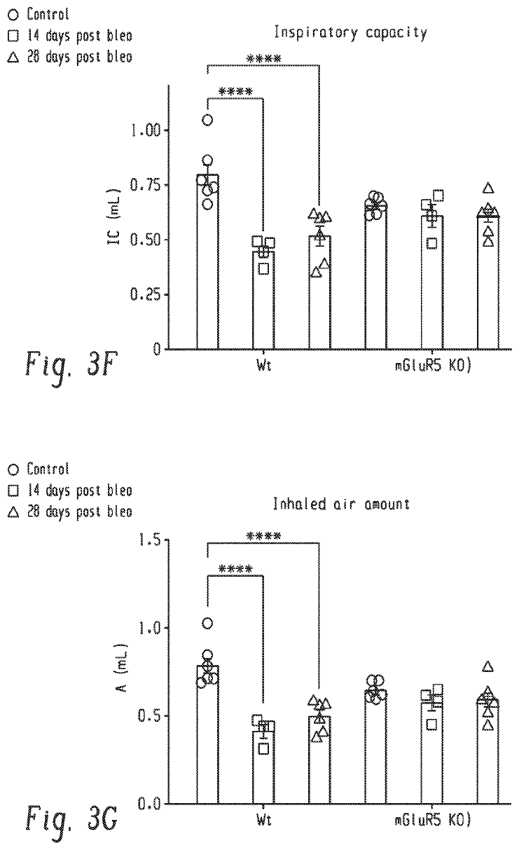

Pulmonary fibrosis development and pulmonary function in wt and mGluR5 KO mice were assessed 14 and 28 days after a single oropharyngeal bleomycin dose (1 U/kg) (FIG. 2A). Deletion of mGluR5 significantly improved survival, as the survival rate was 66% in wt mice while it remained at 100% in mGluR5 KO mice (FIG. 2B). Importantly, deletion of mGluR5 protected against pulmonary fibrosis development compared to the wt littermates, as measured by the level of hydroxyproline in the lungs, which is a biochemical marker of fibrosis (FIG. 2C). In the clinic, the only progressive marker of pulmonary fibrosis is the pulmonary function test (PFT). Therefore, the impact of any potential therapeutic target on regulating pulmonary function parameters is important in identifying therapeutic targets for pulmonary fibrosis (PF). Accordingly, pulmonary function parameters in mice were measured using FlexiVent (SCIREQ). Deletion of mGluR5 protected against bleomycin-induced decline in pulmonary function parameters such as pressure-volume loop (FIG. 3A), tissue stiffness (FIG. 3B), peripheral airway resistance (FIG. 3C), forced vital capacity (FIG. 3D), air flow (FIG. 3E), inspiratory capacity (FIG. 3F) and inhaled air amount (FIG. 3G). These results demonstrate that overactivity of mGluR5 contributes to the development of pulmonary fibrosis.

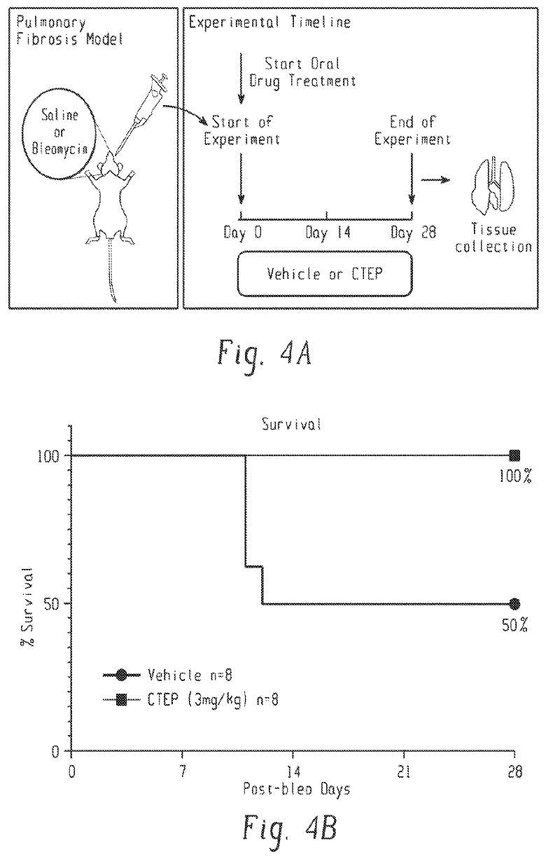

The therapeutic potential of mGluR5 antagonism in PF was evaluated. The efficacy of a selective inhibitor of mGluR5 activation, CTEP, was evaluated in the bleomycin-induced PF mouse model. Daily oral administration of CTEP at 3 mg/kg dose for 28 days (FIG. 4A), which is a maximally effective dose for mGluR5 antagonism [Lindemann et al., J. Pharmacol. Exp. Ther., 339:474-486 (2011)], significantly improved survival, as 100% of the CTEP-treated mice survived compared to the vehicle-treated mice, which had a 50% survival rate (FIG. 4B).

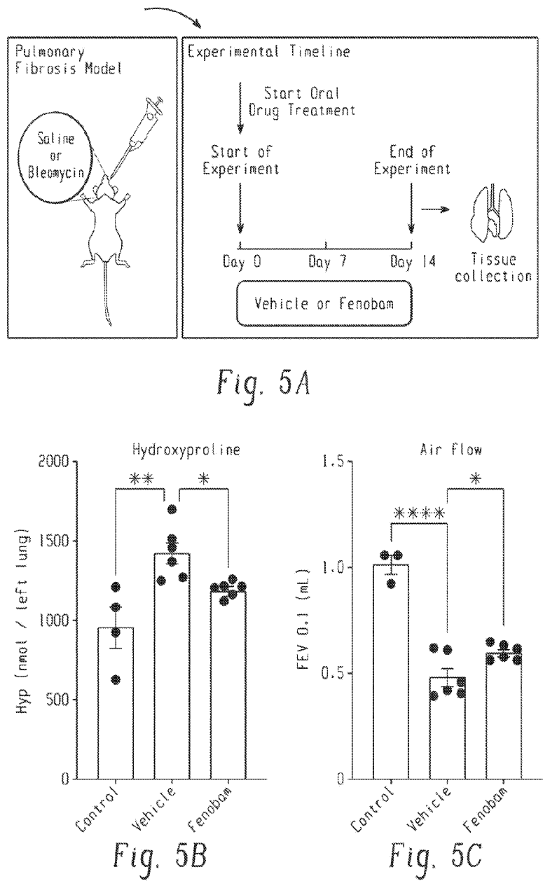

A negative allosteric modulator of mGluR5, fenobam, was tested in the bleomycin-induced PF mouse model. Daily oral administration of fenobam at 30 mg/kg dose (FIG. 5A), which is a maximally effective dose for mGluR5 inhibition [Porter et al., J. Pharmacol. Exp. Ther., 315:711-721 (2005)], for 14 days significantly attenuated fibrosis and decline in a pulmonary function parameter as measured by hydroxyproline level (FIG. 5B) and forced expiratory volume (FEV) at 0.1 sec. (air flow) (FIG. 5C), respectively.

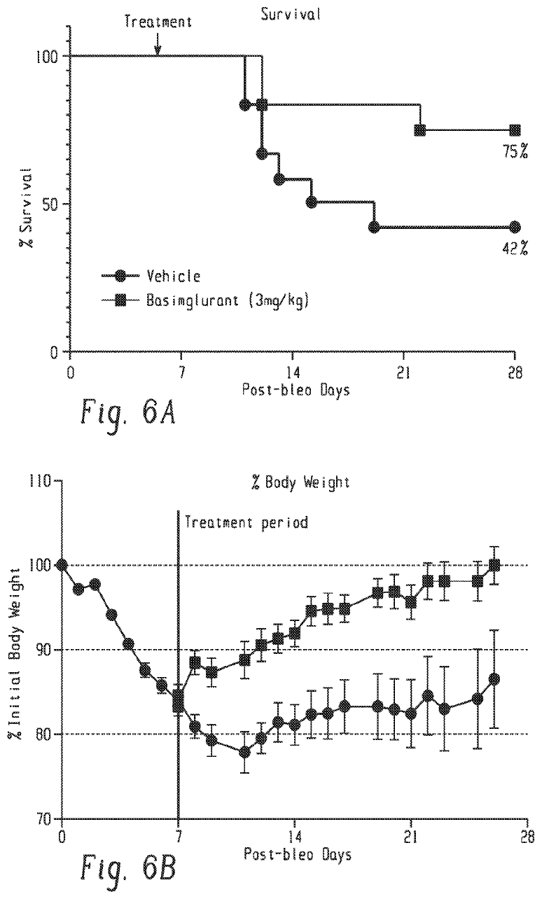

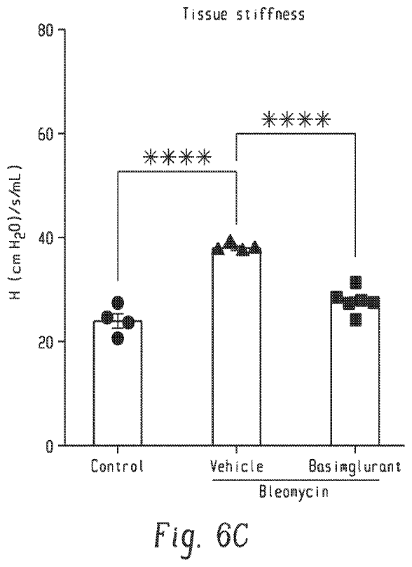

Another negative allosteric modulator of mGluR5, basimglurant, was tested. Treatment with basimglurant commenced once fibrosis was established at 7 days post-bleomycin (FIG. 6). 3 mg/kg oral treatment with basimglurant between 8 and 28 days after a single bleomycin administration significantly attenuated bleomycin-induced PF as measured by increased survival rate (FIG. 6A), reduced body weight loss (FIG. 6B), and reduced pulmonary tissue stiffness (FIG. 6C).

In conclusion, metabotropic glutamate receptor 5 (mGluR5) expression and the level of its endogenous agonist glutamate significantly increased in fibrotic lungs in a murine model of pulmonary fibrosis (PF), indicating that overactivity of mGluR5 signaling in lungs contributes to PF pathology. Either genetic deletion of mGluR5 or pharmacologic inhibition of mGluR5 significantly attenuated bleomycin-induced PF development and significantly increased the survival rate. Therefore, fibrosing lung diseases can be treated through mGluR5 antagonism.

### Example 2. Identification of a Combination Therapy for Pulmonary Fibrosis

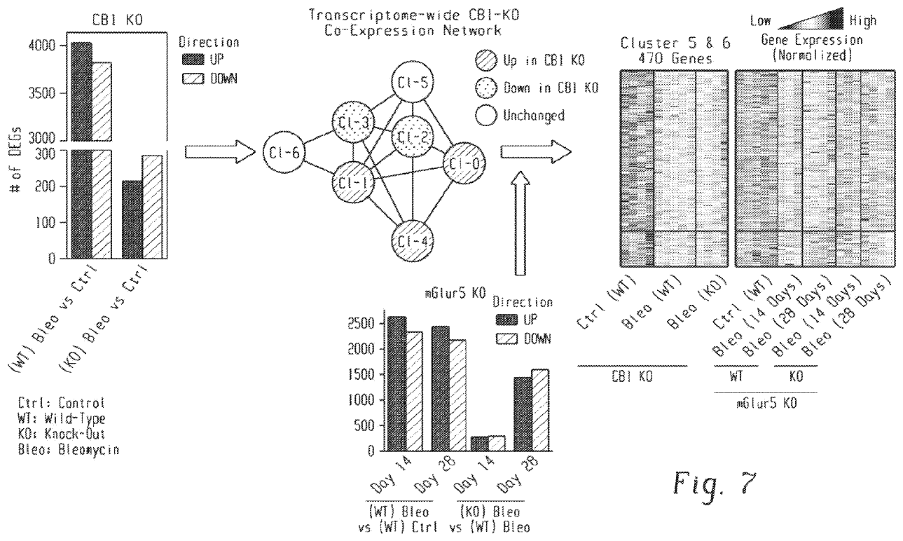

A systems biology approach was used to identify a rational combination therapy for pulmonary fibrosis. Using the bleomycin-induced pulmonary fibrosis (PF) murine model, transcriptomics changes in PF in mice were investigated by conducting lung transcriptomics from healthy control lungs and fibrotic lungs, which were collected 14 days after a single oropharyngeal dose of bleomycin (1 U/kg). Seven differentially expressed gene network clusters (clusters 0-6) in fibrotic murine lungs were identified by transcriptomics analyses (middle part of FIG. 7). The goal of rational combination therapy is to attenuate the pathological alterations in the majority or all of the identified network clusters in the lung transcriptome. Antagonism of cannabinoid receptor 1 (CB1R) was previously identified as a potential therapeutic approach in a different form of PF [Cinar et al., JCI Insight, 2(8):e92281 (2017); Bronova et al., Am. J. Respir. Cell Mol. Biol., 53(4):555-562 (2015); and Cinar et al., Clin. Transl. Med., 11:e471 (2021)]. CB1R antagonism has also been identified as a potential therapeutic approach in fibrosis of the liver, kidneys, heart, lungs and skin [Cinar et al., Pharmacol. Ther., 208:107477 (2020)]. CB 1R-mediated changes in the transcriptome in the bleomycin-induced PF mouse model were investigated using CB1R knockout mice. Deletion of CB1R attenuated the dysregulation in 5 of the 7 gene network clusters (clusters 0, 1, 2, 3 and 4), while attenuating fibrosis development (middle part of FIG. 7).

In addition, mGluR5-mediated changes in the transcriptome in the bleomycin-induced PF mouse model were investigated by comparing wt and mGluR5 KO mice. As a complement to CB1R antagonism, deletion of mGluR5 resulted in normalization of the other two clusters (clusters 5 and 6) (right part of FIG. 7).

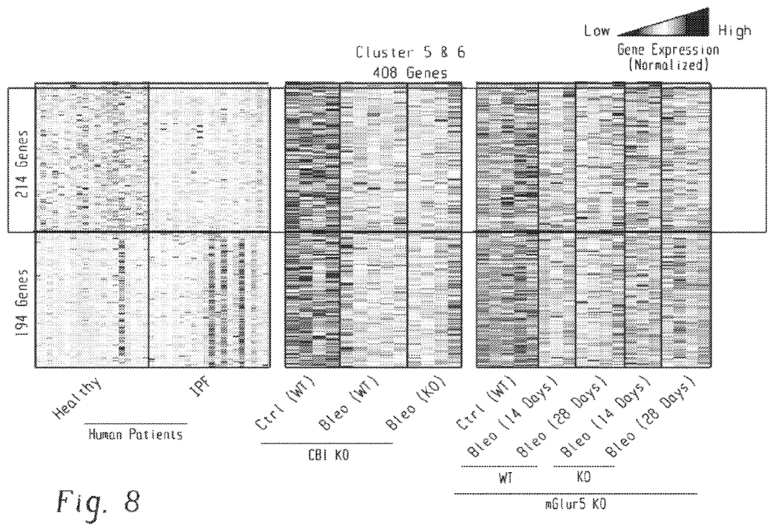

To evaluate the translational value of these findings, the publicly available RNAseq dataset was investigated to determine whether similar transcriptomics changes in fibrotic lungs in mice also exist in human IPF. The genes of clusters 5 and 6 are also downregulated in human IPF compared to healthy lungs (FIG. 8), which was not altered by deletion of CB1R in mice. However, deletion of mGluR5 normalized the pathological transcriptomics changes in mice (FIG. 8).

In conclusion, the combination of a mGluR5 antagonist and a CB1R antagonist (e.g., a peripheral CB1R antagonist) was identified as a therapy for fibrosing lung diseases because the combination attenuated all major pathological alterations in fibrotic lung transcriptome.

It is understood that, while particular embodiments have been illustrated and described, various modifications may be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

1. A method for reducing fibrosis in a pulmonary fibrotic disease in a subject, comprising decreasing fibroproliferative activity at a fibrotic lesion in the subject by reducing the activity of metabotropic glutamate receptor 5 (mGluR5).

2. The method of claim 1 wherein the activity of mGluR5 is reduced by inhibiting gene expression of mGluR5.

3. The method of claim 1, wherein the activity of mGluR5 is reduced by inhibiting production of mGluR5 at the protein stage.

4. The method of claim 1, wherein the mGluR5 activity is reduced by pharmacologic inhibition of mGluR5.

5. The method of claim 4, wherein the pharmacologic inhibition of mGluR5 is by a mGluR5 antagonist administered to the subject.

6. The method of claim 5, wherein the mGluR5 antagonist is a negative allosteric modulator of mGluR5 glutamate signaling or a selective mGluR5 activation inhibitor.

7. The method of claim 6, wherein the negative allosteric modulator of mGluR5 glutamate signaling is fenobam, basimglurant, raseglurant or dipraglurant, or a pharmaceutically acceptable salt, polymorph, isomer or prodrug thereof.

8. The method of claim 6, wherein the selective mGluR5 activation inhibitor is CTEP (2-chloro-4-((2,5-dimethyl-1-(4-(trifluoromethoxy)phenyl)-1H-imidazol-4-yl)ethynyl)pyridine), mavoglurant, auglurant, or remeglurant, or a pharmaceutically acceptable salt, polymorph, isomer or prodrug thereof.

9. The method of claim 5, further comprising administering to the subject a peripheral CB1R antagonist, wherein the peripheral CB1R antagonist is optionally zevaquenabant (MRI-1867), MRI-1891 or TM-38837, or a pharmaceutically acceptable salt, polymorph, isomer or prodrug thereof.

10. A method for treating a fibrotic disease in a subject, comprising administering to the subject in need of treatment a therapeutically effective amount of an antagonist of mGluR5 in combination with a therapeutically effective amount of an antagonist of CB1R, wherein the mGluR5 antagonist is a negative allosteric modulator of mGluR5 glutamate signaling or a selective mGluR5 activation inhibitor, and wherein the CB1R antagonist is a peripheral CB1R antagonist, or a pharmaceutically acceptable salt, polymorph, isomer, or prodrug thereof.

11. The method of claim 10, wherein:
the negative allosteric modulator of mGluR5 glutamate signaling is fenobam, basimglurant, raseglurant or dipraglurant;
the selective mGluR5 activation inhibitor is CTEP (2-chloro-4-((2,5-dimethyl-1-(4-(trifluoromethoxy)phenyl)-1H-imidazol-4-yl)ethynyl)pyridine), mavoglurant, auglurant, or remeglurant; and
the peripheral CB1R antagonist is zevaquenabant (MRI-1867), MRI-1891 or TM-38837; or
a pharmaceutically acceptable salt, polymorph, isomer or prodrug thereof.

12. The method of claim 1, wherein the pulmonary fibrotic disease or the fibrotic disease is selected from pulmonary fibrosis, chronic obstructive pulmonary disease (COPD), idiopathic pulmonary fibrosis (IPF), Hermansky-Pudlak syndrome pulmonary fibrosis (HPSPF), interstitial lung disease (including usual interstitial pneumonia and scleroderma-related interstitial lung disease [Ssc-ILD]), respiratory bronchiolitis/interstitial lung disease, hypersensitivity pneumonitis, primary pulmonary hypertension (including prevention of the formation of plexiform lesion), chronic graft versus host disease (cGVHD), hepatic/liver fibrosis, cirrhosis, non-alcoholic steatohepatitis (NASH), cardiac fibrosis, myocardial fibrosis (e.g., interstitial fibrosis, subepicardial fibrosis, subendocardial fibrosis and replacement fibrosis), cardiomyopathy, congestive heart failure, renal fibrosis, chronic renal disease, diabetic nephropathy, retroperitoneal fibrosis (Ormond's disease), nephrogenic systemic fibrosis, Schistosoma mansoni infection, herpes virus-associated diseases (including lung and dermatological manifestations), SARS-CoV-2- and variants thereof-related pulmonary diseases, alcohol use disorder (AUD)-related lung injury, acute respiratory distress syndrome (ARDS), synthetic cannabinoids-induced lung injury and respiratory failure, keloid scarring, lupus, nephrogenic fibrosing dermopathy, fibrosing lesions associated with Schistosoma japonicum infection, autoimmune diseases (e.g., rheumatoid arthritis), pathogenic fibrosis, Lyme disease, stromal remodeling in pancreatitis, stromal fibrosis, uterine fibroids, ovarian fibrosis, corneal fibrosis, ischemia-related conditions including pre-ischemic and post-ischemic conditions (e.g., congestive heart failure), post-surgical scarring (including abdominal adhesions), and wide angle glaucoma trabeculotomy.

13. The method of claim 5, wherein the mGluR5 antagonist or/and the peripheral CB1R antagonist is/are administered at a dose of about 0.01 mg/kg to 100 mg/kg, 0.1 mg/kg to 50 mg/kg, 0.1 mg/kg to 20 mg/kg, 0.1 mg/kg to 10 mg/kg, 0.1 mg/kg to 100 mg/kg, 1 mg/kg to 100 mg/kg, or 10 mg/kg to 100 mg/kg daily.

14. The method of claim 5, wherein the mGluR5 antagonist or/and the peripheral CB1R antagonist is/are administered orally, parenterally (e.g., intravenously, subcutaneously or intramuscularly), intranasally, sublingually, or buccally, or by oral or nasal inhalation.

15. The method of claim 1, wherein the subject is a mammal, such as a human.

16. A pharmaceutical composition comprising:
(i) a peripheral CB1R antagonist, and
(ii) a mGluR5 antagonist, and
(iii) a pharmaceutically acceptable excipient or carrier.

17. The pharmaceutical composition of claim 16, wherein the pharmaceutical composition is effective for treating a fibrotic disease when administered to a subject in need thereof.

18. The pharmaceutical composition of claim 16, wherein the pharmaceutical composition is administered orally, parenterally (e.g., intravenously, subcutaneously or intramuscularly), intranasally, sublingually, or buccally, or by oral or nasal inhalation.

19. The pharmaceutical composition of claim 16, which is formulated for administration to a mammal.

20. The pharmaceutical composition of claim 19, wherein the mammal is a human.
